(19) United States
(12) Patent Application Publication
(10) Pub. No.: US 2023/0078423 A1
(43) Pub. Date: Mar. 16, 2023

(54) THREE-DIMENSIONAL CELL CULTURE

(71) Applicant: LifeTaq-Analytics GmbH, Tulln av der Donau (AT)

(72) Inventors: Manfred TASCHNER, Wien (AT); Volker LORBER, Tulln an der Donau (AT)

(21) Appl. No.: 17/798,013

(22) PCT Filed: Feb. 5, 2021
(86) PCT No.: PCT/EP2021/052816

§ 371 Date: Aug. 5, 2022

(30) Foreign Application Priority Data
Feb. 5, 2020 (EP) 20155628.9

(51) Int. Cl. C12M 1/32; C12M 1/00; C12M 1/12

(52) U.S. Cl. CPC C12M 23/12; C12M 23/52; C12M 25/04; C12M 29/10; C12N 2513/00

(57) ABSTRACT

The invention relates to a cell culture device for a three-dimensional cell culture with one or more cell culture units, characterized in that a cell culture unit comprises:
i. at least one matrix holder, with a central opening for uptake of a matrix;
ii. a support, which is fixedly or reversibly connected to the at least one matrix holder; and
iii. a well strip, consisting of a vessel with at least one cavity, which can comprise at least one matrix holder up to the upper end of the central opening or more,
   wherein the matrix holder is vertically oriented.

### FIELD OF INVENTION

The present invention relates to the technical field of cell culture, in particular to three-dimensional cell culture of animal and human cells.

### BACKGROUND OF THE INVENTION

Animal and human cells are traditionally cultivated in the second dimension in biomedical and pharmaceutical research. Thereby, cells are transferred to a plastic or glass surface, on which they can attach and proliferate. This environment, however, does not correspond to the natural, physiological conditions of the cells and therefore very often leads to loss of function, dedifferentiation of the cells and in the worst case to cell death. (DE102007020302A1). Cells in the human body behave fundamentally differently in their milieu than those in culture. The reasons are complex intercellular as well as extracellular processes, which are influenced for example by neighbouring cells, the tissue or also by soluble factors, such as growth factors or hormones. 2D cell cultures allow the attachment and growth of cells outside an organism, but do not adequately reflect the natural growth conditions of cells in their natural environment.

To reduce these gaps between two-dimensional cell culture and the in vivo milieu, numerous three-dimensional cell culture systems have been developed as alternatives over the past decade. 3D cultures bring numerous advantages, as the cultivated cells allow for a more physiologically authentic model of human tissue. These include for example systems with solid supports, such as porous support structures, which mimic the extracellular matrix, and support-independent systems, such as spheroid cultures. Currently approximately 150 different 3D culture systems exist worldwide, of which 76% use a scaffold for cultivation (scaffold-based) (BCC Research: “3D Cell Cultures: Technologies and Global Markets,” (BIO140A), Publ. Date: January 2015). Examples are hanging-drop multiwell plates, which are used to produce spheroids (US 2013/236924 A1), systems for generating three-dimensional matrices, which comprise a magnetic core (US 2017/137766 A1) or for example a cell culture system, which comprises a three-dimensional biocompatible scaffold structure and nanoparticles. These structures shall mimic the extracellular matrix as natural environment of the cells (DE102007020302A1).

DE10003521A1 describes a device for the production of three-dimensional matrix bodies, which consist of a support substance and therein embedded cell cultures, and their use for cultivation of cells in multiwell plates.

US 2019/276784 A1 discloses different complex cell culture devices, which shall mimic the joint of a mammal. The cell culture device comprises several hollow scaffolds with many openings, for uptake of a matrix in the cavity of the scaffold.

US 2007/237683 A1 discloses a device, which serves the improved optical imaging of two-dimensional cell cultures. The device comprises a multiwell plate with cylindrical inlets without bottom, which hold the well strips. The well strips also comprise cylindrical cavities, which fit into the inlets of the multiwell plate, but have a transparent bottom, on which the cells are cultivated.

CA 2579680 A1 discloses a bioreactor with a perfusion unit and a multiwell plate, which comprises a plurality of bioreactor units, in which different experiments can be carried out in each case.

WO 2004/027016 A1 discloses a perfusion incubator with a peristaltic pump, which can be used in particular for raising embryos.

The handling of scaffold-based systems and already existing three-dimensional cell culture systems however is often difficult and an automation of the cell culture is very difficult if not impossible. Therefore, there is a need for three-dimensional cell culture systems, which are robust and easy to handle and preferably can also be run in an automated system.

### SUMMARY OF THE INVENTION

It is the object of the present invention to provide improved devices for in vitro cultivation of animal and human cells. In particular, it is an object of the present invention to provide cell culture devices, which allow a simplified handling and whose handling can optionally also be automated.

This object is solved by the subject matter of the invention.

The use of scaffold-based matrices in automated cell culture processes proves to be difficult due to the complexity with respect to different materials and techniques. For this reason, a new cell culture system was developed in which hydrogels as well as porous support structures can be used. In particular, a cell culture device in vertical orientation has been developed, with which cells in a three-dimensional (3D) environment can be cultivated. The goal herein is to reduce the manual effort as much as possible and to open up possibilities for 3D cultures from hydrogels or porous support structures for medium- to high-throughput applications.

The present invention comprises a cell culture device for a three-dimensional cell culture with one or more cell culture units, characterized in that a cell culture unit comprises the following:

i. at least one matrix holder (12), with a central opening (13) for uptake of a matrix;

ii. a support (10), which is fixedly or reversibly connected to the at least one matrix holder (12); and

iii. a well strip (11), consisting of a vessel with at least one cavity, which can comprise at least one matrix holder (12) up to the upper end of the central opening (13) or more,

wherein the matrix holder (12) is vertically oriented.

The compact design of the cell culture unit allows for easy handling not only of one, but also of several matrix holders.

The central opening (13) of the matrix holder (12) can comprise a continuous opening (13a and 13c) or a cavity with a wall (13b).

The inner walls of the opening (13) can comprise an angle of 1-30°, so that the two ends of the opening (13) can have a different diameter. In particular, they can comprise an angle of about 1, 2, 3, 4, 5, 6, 7, 8, 9, 10, 11, 12, 13, 14, 15, 16, 17, 18, 19, 20, 21, 22, 23, 24, 25, 26, 27, 28 or 29°.

The present invention also comprises a colonization device (30, 33).

In one embodiment of the matrix holder (12), the opening (13) of the matrix holder comprises a cavity (13c) for sealing and reversible attachment to a colonization device (30, 33).

According to a particular embodiment, the colonization device (30, 33) comprises sealing elements (31). The colonization device can comprise elevations (32).

In a further embodiment, the opening (13a) comprises an upper cavity (13d), to avoid the entrapment of air bubbles in the matrix during horizontal colonialization.

According to a preferred embodiment of the cell culture device, the central opening (13) comprises a matrix, which is a solid porous support or a hydrogel, or a combination thereof. For example, the matrix can be a solid porous support, to which a hydrogel is applied and/or which is filled with a hydrogel.

A cell culture unit can comprise several matrix holders (12). In particular, the cell culture unit can comprise 2, 3, 4, 5, 6, 7, 8, 9, 10, 11, 12, 13, 14, 15, 16, 17, 18, 19 or 20 matrix holders (12). The matrix holders (12), comprised by a cell culture unit, can include the same or different matrices.

In particular, the matrix holders (12) of a cell culture unit each comprise a matrix of hydrogel or each a matrix, which is a solid porous support. This allows simultaneous culture of cells in the same three-dimensional environment, wherein the medium in the wells of the well strips (11) can be identical or different. On the one hand, the composition of the medium can be different, for example, to study the influence of the medium on cell growth. On the other hand, if the medium contains a test agent, different agents or different concentrations of an agent can be tested.

In particular, a cell culture unit comprises matrix holders (12) whose matrix is a hydrogel and matrix holders whose matrix is a solid porous support. If a cell culture unit comprises different matrix holders (12), this enables, for example, a rapid screening of different three-dimensional environments.

In particular, the matrix consists of natural, semi-synthetic or synthetic material or a combination thereof. Optionally, the matrix cab be modified or coated with protein or peptide sequences. For example, the matrix can comprise antibodies or antibody fragments or RGD sequences.

In particular, any material which is compatible with the culture of animal or human cells, can be used as material for the herein described matrix. In particular, the natural material is selected from collagen, fibrin, glycosamine glycans and hyaluronic acid, and the synthetic or semi-synthetic material is selected from polymeric components such as polymethyl methacrylate, polylactic acid, polyglycol, polystyrene and ceramic components such as hydroxyapatite or tricalcium phosphate.

According to a further embodiment, the support (10) comprises an element (15) for uptake of the matrix holder (12) and the matrix holder (12) comprises notches (14a, 14b) at its upper end, via which it can be reversibly connected to the element (15) of the support (10).

According to a further particular embodiment of the support (10) of the cell culture device, the support comprises at least one end, preferably at both ends, an opening for fastening a magnetizable nut (45), for example by means of a screw. In particular, the support comprises additional multiple openings for matrix holders (46), which can be connected to the support, for example by means of fastening pins (47).

According to a particular embodiment, the device additionally comprises a transport unit (20), which can transport one or more cell culture units (21).

In particular, the transport unit (20) facilitates the use of the present cell culture device in an automated environment. By means of the transport unit, one or more cell culture units can be moved and operated. In particular, this enables the automation of certain processes, such as automated media change or automated colonialization of the units. In particular, the automation can take place by means of a robot.

According to a particular embodiment, the transport unit comprises:

i. a support rail (22) for attachment of one or more cell culture units (21) to the transport unit (20),

ii. an uptake unit (20a) for a support rail (22),

iii. a cover (23), and

iv. two spacer elements (24), which are optionally directly connected to each other, for uptake in an orderly manner of at least two or more supports (10) of a matrix holder,

v. optionally at least one identification element (26) such as for example a RFID chip or a barcode, and

vi. optionally a centering unit (27).

In particular, the well strip (11) comprises a position (25) for attachment to the support rail (20).

The spacer elements serve for the uptake and orderly array of the cell culture units, which are mounted in the spacer above the support (10) of the matrix holders. In particular, the cell culture units are arranged in parallel in the spacer (24). The spacer elements can be fixedly or reversibly connected to each other or also not be directly connected to each other, but only via the supports of the matrix holders.

According to a particular embodiment, the transport unit (20) comprises:
i) a plate (48), which forms the bottom of the transport unit,
ii) openings in the plate (48) for centering the transport unit on the plate (49),
iii) openings in the plate (48) for screw fittings (50),
iv) a further plate (51), which is mounted approximately at the middle height of the transport unit,
v) magnetizable metal bars (52),
vi) a centering support (54),
vii) openings in the plate (51) for the centering holder (54),
viii) and a centering object (55).

In particular, the magnetizable metal bars (52) serve for the reversible connection with a cell culture robot. According to a specific example, the magnetizable metal bars (52) serve as docking point for reversible connection with a robotized unit for transport of the transfer unit, for example for reversible connection with magnets of the Oli-MAT (available from LifeTaq-Analytics GmbH, Tulln an der Donau). The Z-axis of the Oli-MAT comprises two permanent magnets, with whose help the transfer unit can be carried from position to position. By means of a short impulse, the magnets can be demagnetized for a short time, which leads to the stopping of the transfer unit.

The present invention also comprises a method of cell cultivation using the herein disclosed cell culture device.

In particular, the method comprises the following steps:

i. suspending the cells to be cultivated in an appropriate amount of a cell culture medium to form a cell suspension,

ii. colonializing the matrix in the opening (13) of the matrix holder (12) with the cell suspension,

iii. transferring the matrix holder into a well strip (11), in particular using the support of the matrix holder (10), and

iv. incubating the cell culture device, which contains the cells, under conditions that allow cell growth.

According to a particular embodiment of the herein disclosed method, the colonialization of the matrix with the cell suspension takes place in a horizontal arrangement of the matrix holder (12).

In particular, the matrix holder (12) of the cell culture device for use in this method, comprises an opening (13) with a cavity (13c), wherein the matrix holder is reversibly connected via a complementary sealing element (31) on a colonization device (30) to such, wherein the used matrix is a hydrogel and the cell cultivation method comprises in particular the following steps:

i. suspending the cells to be cultivated in an appropriate amount of a cell culture medium to form a cell suspension,

ii. mixing the cell suspension with one or more components of a hydrogel, to produce a cell suspension-hydrogel mixture,

iii. colonializing the opening (13) of the matrix holder (12), which is horizontally aligned in the colonization device (30), with the cell suspension-hydrogel mixture,

iv. incubating the cell batch in a suitable cultivation environment, preferably at 37° C. and 5% CO₂, until polymerization of the matrix,

v. separating the matrix holder (12) from the colonization device (30),

vi. transferring the matrix holder (12) into a well strip (11) filled with medium, preferably using the support of the matrix holder (10),

vii. incubating the cell culture device, which contains the cell suspension-matrix mixture, under conditions that allow cell growth.

The polymerization of the hydrogel can take in particular about 5 minutes and up to 24 hours. The duration of the polymerization is typically dependent on the composition of the hydrogel, in particular its components and their concentration, as well as on the environment, for example the humidity and the air temperature. Accordingly, the person skilled in the art is able to determine the time required for hardening of the gel.

After polymerization of the cell suspension-hydrogel mixture, a further layer of a cell suspension-hydrogel mixture can be introduced into the matrix. The further cell suspension-hydrogel mixture can contain other cells than the first mixture, such as modified variants of the cells of the first mixture. The further cell suspension-hydrogel mixture can contain the same cells as the first mixture, but for example a different hydrogel composition.

According to a particular embodiment, the colonization device (30) comprises an inner elevation (32), which ensures, that during polymerization of the cell suspension-hydrogel mixture, a cavity is formed in the opening (13), which can be filled with the further cell suspension-hydrogel mixture after removal of the colonization device (30). This allows for example the co-culture of different cell types, or the culture of one cell type in different three-dimensional environments in a matrix holder (12).

According to a further embodiment of the herein disclosed method, wherein the colonialization of the matrix with the cell suspension takes place in a horizontal arrangement of the matrix holder (12), the opening (13) of the matrix holder (12) comprises a cavity with a wall (13b), and the matrix is a solid porous support. In particular, the method comprises the following steps:

i. transfer of a solid porous support into the cavity (13b),

ii. suspending the cells to be cultivated in an appropriate amount of a cell culture medium to form a cell suspension,

iii. transferring the cell suspension into the opening (13b) of the matrix holder (12),

iv. incubating the colonization device for at least 30 min to 24 h under conditions, that allow the cells to grow on the solid porous support,

v. transfer of the matrix holder into a well strip (11) previously filled with medium, in particular using the support of the matrix holder (10), and

vi. incubating the cell culture device, which contains the cells attached to the porous support, under conditions that allow cell growth.

According to a further embodiment of the herein disclosed method, the colonialization of the matrix with the cell suspension take place in a vertical arrangement of the matrix holder (12).

In particular, the matrix holder (12) of the cell culture device for use in this method, comprises an upper opening (13d), the matrix holder (12) is reversibly connected to a colonization device (33) and the matrix is a hydrogel. In particular, the method comprises the following steps:

i. suspending the cells to be cultivated in an appropriate amount of a cell culture medium to form a cell suspension,

ii. mixing the cell suspension with a hydrogel, to produce a cell suspension-hydrogel mixture,

iii. filling the cavities (wells) of the colonization device (33) with the cell suspension-hydrogel mixture,

iv. colonializing the opening (13) of the matrix holder by immersing the matrix holder (12) in the colonization device (33),

v. incubating the cell batch in a suitable cultivation environment, preferably at 37° C. and 5% CO₂, until polymerization of the matrix,

vi. transferring the matrix holder (12) from the colonization device (33) into a well strip (11) filled with culture medium, and

vii. incubating the cell culture device, which contains the cell suspension-matrix mixture, under conditions that allow cell growth.

To minimize the volume, the volume of the wells of the colonization device (33) can be reduced in contrast to the well strip (11). The colonization device (33) can additionally comprise filling elements (40). The wells of the colonization device (33) can be filled with medium via the filling elements (40).

In particular, the method comprises the following steps, when a filling device (33) is used, comprising filling elements (40):

i. the cells to be cultivated in an appropriate amount of a cell culture medium to form a cell suspension,

ii. mixing the cell suspension with a hydrogel, to produce a cell suspension-hydrogel mixture,

iii. inserting the matrix holder into the colonization device (33),

iv. filling the wells of the colonization device (33) with the cell suspension-hydrogel mixture via the filling elements (40), where the opening (13) of the matrix holder is colonialized with the cell suspension-hydrogel mixture,

v. incubating the cell suspension in a suitable cultivation environment, preferably at 37° C. and 5% 5% CO₂, until polymerization of the matrix

vi. transferring the matrix holder (12) from the colonization device (33) into a well strip (11) filled with culture medium, and

vii. incubating the cell culture device, which contains the cell suspension-matrix mixture, under conditions that allow cell growth.

According to a further particular embodiment, the matrix holder is oriented horizontally for colonialization. In this embodiment, the opening (13) of the matrix holder (12) comprises a cavity with a wall (13b) and the matrix is a solid porous support. The method for colonialization of the matrix holder in this embodiment comprises the steps:

i. transferring a solid porous support into the cavity (13b),

ii. suspending the cells to be cultivated in an appropriate amount of a cell culture medium to form a cell suspension,

iii. transferring the cell suspension into the opening (13b) of the matrix holder (12),

iv. incubating the colonization device, in particular for at least 30 min to 24 h, under conditions, that allow the cells to grow on the solid porous support.

Subsequently, the matrix holder can be transferred into a well strip (11) filled with cell culture medium, in particular using the support of the matrix holder (10), and the cells can be incubated in the cell culture device, which contains the cells attached to the porous support, under conditions that allow cell growth.

According to a particular embodiment of the methods of the invention, the transfer of the matrix holders into a well strip, the transfer of an entire cell culture device and/or the transfer of a well strip can take place automated by a robotic gripper arm.

According to a particular embodiment of the cell culture device, the well strip is a perfusion device (34), which comprises tube connections (41) for connection to a pump. Via these tube connections by means of a pump a continuous flow of a cell-compatible fluid, such as cell culture medium, can be directed through the perfusion device.

According to a further embodiment, the cell culture device further comprises a box (44) and an insert (42) for uptake of one or more cell culture units (21) in the box (44).

According to a further embodiment, the cell culture device further comprises a box (44) and an insert (43) for uptake of one or more colonization devices (33) in the box (44).

### FIGURES

FIG. 1 shows components of one embodiment of the cell culture unit (21), in particular matrix holders (12) with various openings (13a, 13b, 13c, 13d), a well strip (11) and a support (10).

FIG. 2 shows components of one embodiment of the transport unit (20), in particular an uptake unit (20a), several cell culture units (21), a support rail (22), a cover (23), spacer elements (24), an identification element (26) and a centering unit (27).

FIG. 3 shows colonization devices. FIG. 3a) shows a colonization device (30) with sealing elements (31), which can be reversibly connected to the matrix holder (12) via the cavity of the opening (13c). FIG. 3b) shows a colonization device (30) comprising elevations (32).

FIG. 4 shows components of a cell culture unit and the colonization device (33) with filling elements (40).

FIG. 5 shows components of a cell culture unit and a perfusion device (34) with tube connections (41).

FIG. 6 shows components of a cell culture unit (10, 11, 12) and a box (44) and an insert (42) for uptake of one or more cell culture units in the box.

FIG. 7 shows components of a cell culture unit (10, 12) with a colonization device (30), as well as a box (44) for uptake of one or more colonization devices.

FIG. 8 shows a further embodiment of a support (10) of the cell culture device with an opening for fastening of a magnetizable nut by means of a screw (45), several openings in the support for matrix holders (46), fastening pins of a matrix holder (47) and the matrix holder (12).

FIG. 9 shows a further embodiment of the transport unit (20) comprising several cell culture devices (21). The figure shows a plate (48) which forms the bottom of the transport unit, openings in the plate (48) for centering the transport unit on the plate (49), openings in the plate (48) for screw fittings (50), a further plate (51) which is mounted approximately at the middle height of the transport unit, magnetizable metal bars (52), a centering holder (54) and openings in the plate (51) for the centering holder (54) and a centering object (55).

FIG. 10 shows another embodiment of a colonization device (30) comprising sealing elements (31). The figure shows a support (10) with a matrix holder (12), which is inserted into the colonization device.

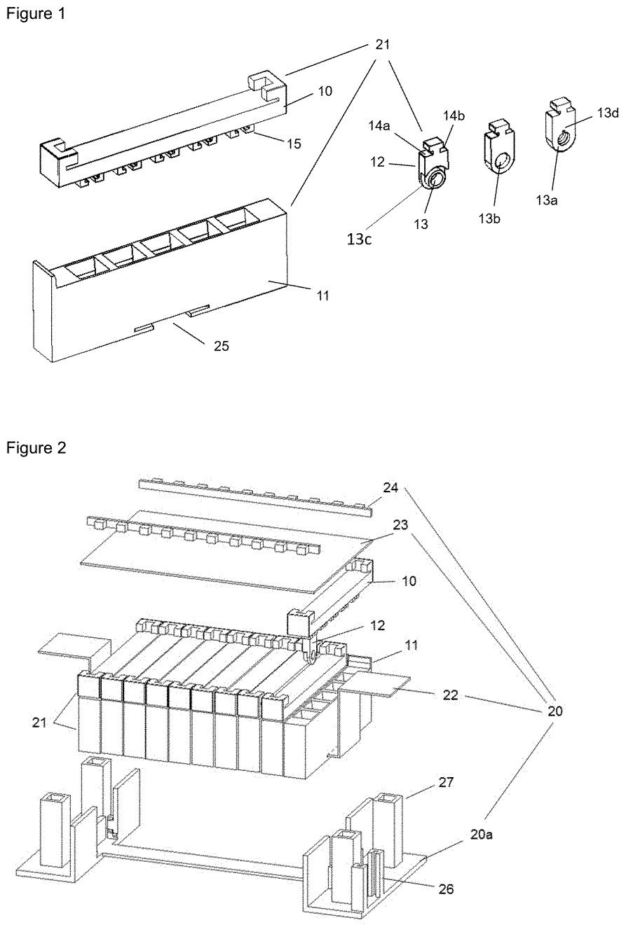

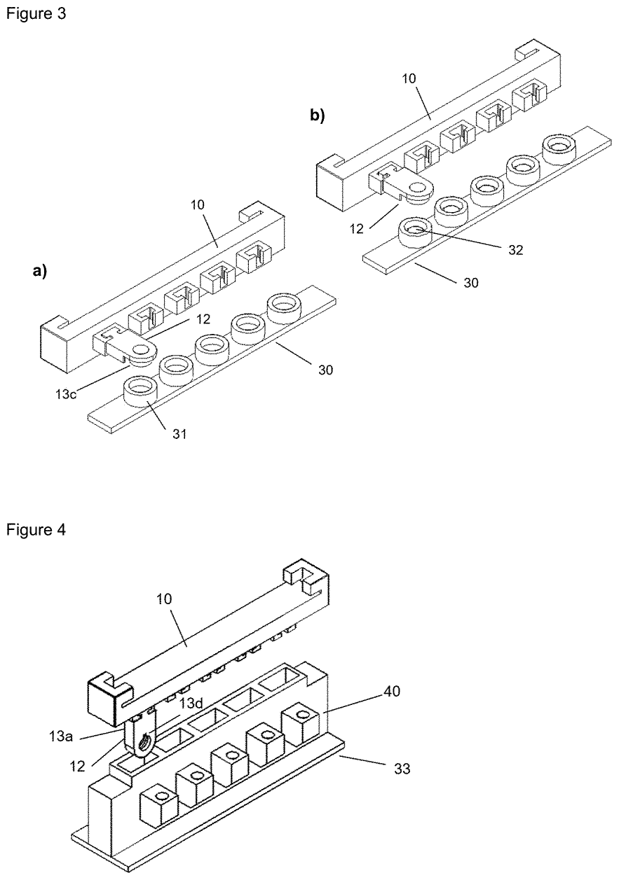

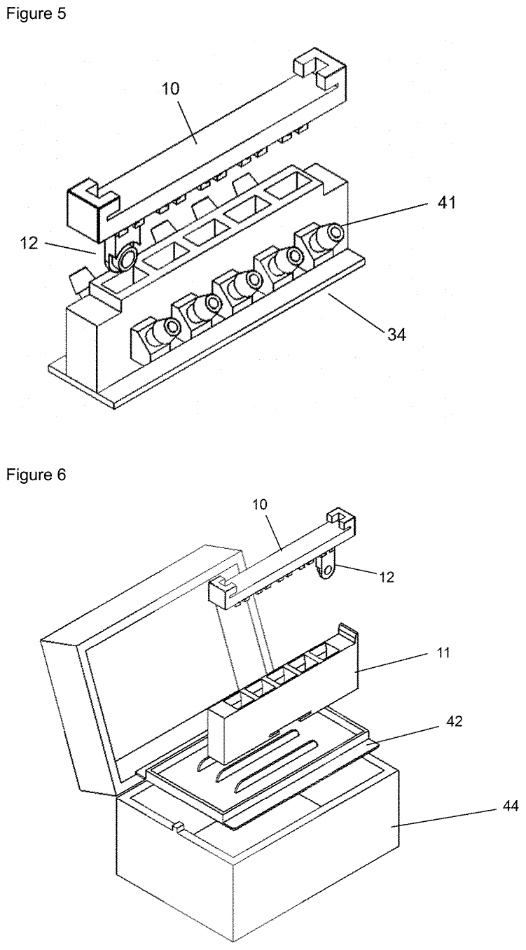

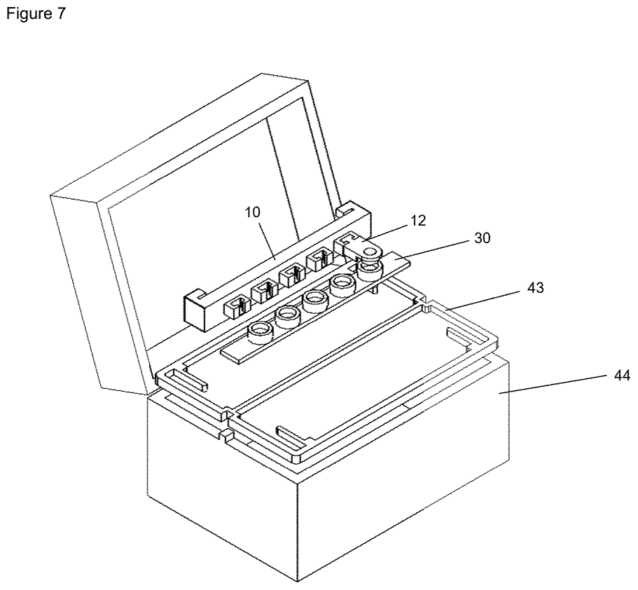

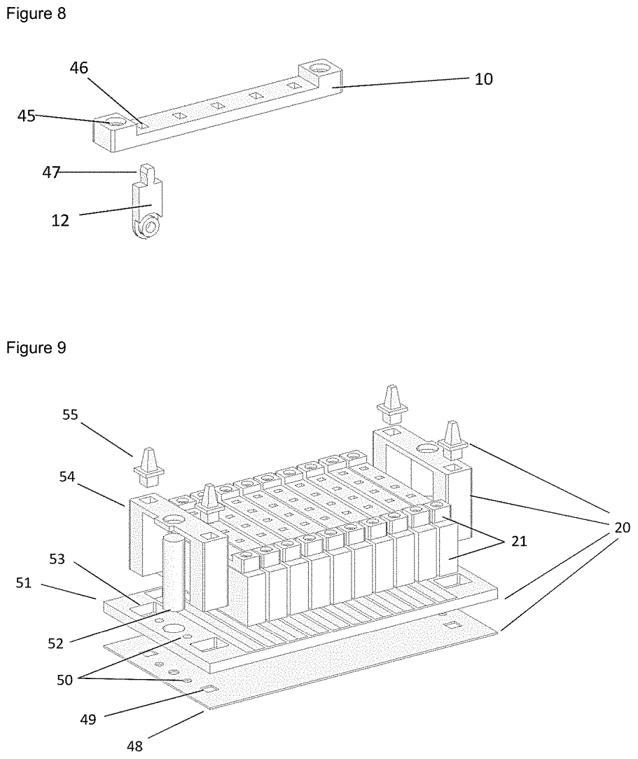

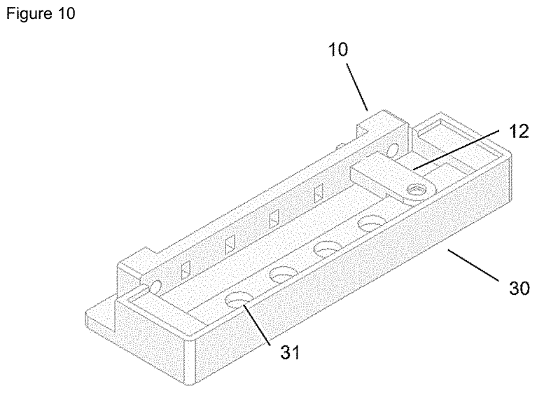

### DETAILED DESCRIPTION

The use of different matrices in automated cell culture processes is difficult due to the complexity due to different materials and techniques. For this reason a system was developed in which hydrogels as well as solid porous supports can be used. The purpose of using automated processes is to minimize the manual effort.

According to the invention, the present cell culture device opens up the possibility to use three-dimensional cell cultures with a matrix of hydrogel and/or porous support structures in automated medium- to high-throughput applications. For example, toxicology studies can thus be performed in an automated environment in a three-dimensional cell culture.

The invention relates to cell culture devices for in vitro cultivation of biological cells and of tissue, and cultivation methods using these cell culture devices. In particular, the present cell culture devices are robust and easy to handle and enable the in vitro cultivation of cells in a three-dimensional environment. Another particular advantage of the present cell culture devices is, that they are suitable for use in an automation-based environment, for example in combination with a robot.

Due to the compact design of the cell culture device according to the invention, in particular the cell culture units, the cultivation of different cell types in different environments can proceed simultaneously, wherein variations in the cultivation of the cells, in particular due to human interaction with the cell culture device, are reduced. In particular, several replicates of a cell culture system can be cultivated simultaneously.

The present cell culture device comprises at least one matrix holder, a support for fixing the matrix holder and a vessel with at least one usually tub-shaped cavity (well), which is called well strip. The cell culture device according to the invention is particularly characterized in that the at least one matrix holder is vertically oriented.

The matrix holder is particularly characterized in that it consists of an outer basic base form and comprises an inner central opening. This opening can have various modifications and shapes, for example it may be a continuous opening or a cavity with a back side wall, which is therefore open to one side only. This allows that hydrogels as well as solid porous supports can be used. The opening of the matrix holder can be continuous or non-continuous, the matrix holder can for example have a rectangular or circular opening and can for example be shaped as cylinder, cuboid, pyramid, or cube.

The inner walls of the opening of the matrix holder can be straight or comprise an angle of about 1 to 25 degrees. In particular, the inner walls of the opening of the matrix holder can have an angle of about 1, 2, 3, 4, 5, 6, 7, 8, 9, 10, 11, 12, 13, 14, 15, 16, 17, 18, 19, 20, 21, 22, 23, 24 or 25 degrees. Optionally, also the outer wall of the opening can have such an angle.

Therefore, the two ends of the opening can have a different diameter. For example, if the opening is a cavity with a wall, the back side wall can thus have a smaller diameter than the front side wall opening. This has the effect, that hydrogels adhere less to the back side wall and can be more easily removed from the opening of the matrix holder. Therefore, damages to the hydrogel and cells during removal from the matrix holder can avoided.

In a particular embodiment, the matrix holder comprises also modifications around the inner central opening. These modifications can for example be further cavities around the opening, creating a wall around the opening. In particular, this wall represents a raised curvature with an inner tub-shaped opening, which optionally comprises a wall, for example a back side wall, or is continuous. Modifications of this type are particularly suitable for reversibly connecting the matrix holder to a corresponding compatible colonization device, for example via a plug-in system.

In another particular embodiment, the matrix holder comprises a cavity or an indentation at the upper edge of the opening, allowing air bubbles to escape from the well. Therefore, for example, the entrapment of air bubbles in the matrix, particularly during colonialization, can be avoided.

It is known that mechanical stimulation stimulates the formation of extracellular matrix proteins. According to a particular embodiment, the matrix holder is formed of soft, elastic silicone of different Shore hardness, allowing mechanical forces, such as pulling, stretching or pushing, to act on the matrix holder and thus on the cells in the matrix without negative effect.

The present cell culture device comprises at least one support, that is fixedly or reversibly connected to at least one matrix holder. Preferably, the support is adapted to fixedly or reversibly take up more than one matrix holder, in particular at least 2, 3, 4, 5, 6, 7, 8, 9, 10, 11, 12, 13, 14, 15, 16, 17, 18, 19, 20, 25, 30, 35, 40, 45, 50 or more matrix holders. Preferably, the matrix holders are thereby arranged in longitudinal direction, in particular in a line or in parallel lines.

According to a particular embodiment, in which matrix holders are reversibly connected to the support, the support comprises constructions or elements for uptake of one matrix holder each. Preferably, these constructions or elements are arranged in longitudinal direction, in particular in a line or in parallel lines. In particular, the matrix holders comprise elements, such as notches, that are compatible with the constructions or elements of the support and can therefore be reversibly connected to the support. For example, the elements of the support and the indentations of a matrix holder can constitute a plug-in system, in that the indentations of the matrix holder are plugged or slid into the elements of the support.

According to another particular embodiment, the support comprises elements for fixed or reversible connection and attachment to a transport unit. In particular, the support and the transport unit comprise compatible elements via which the support and the transport unit can be fixedly or reversibly connected to each other. For example, they can be connected by sliding or inserting one of the elements into the matching element.

According to another particular embodiment of the support of the cell culture device, the support comprises at least one end, preferably both ends, an opening for fastening a magnetizable nut, for example by means of a screw. In particular, the support additionally comprises several openings for matrix holders, that can for example be connected to the support by means of fastening pins.

Preferably, the nut is made of iron or another magnetizable metal. The nut cab be magnetized by permanent magnets, for example in a separate construction such as a robot. The separate construction can for example be a cell culture robot, for example Oli-Mat (available from LifeTaq-Analytics GmbH, Tulln an der Donau). Via the magnetizable nut, the support can be reversibly connected to the separate construction, thus allowing the movement of the cell culture unit (for example into fresh culture medium) over the support. To set down at the destination, the supports can be disconnected from the magnet by physical force or by a short pulse.

The present cell culture device comprises at least one well strip, which consists of a vessel with at least one cavity. This cavity can fully comprise at least one matrix holder, at least up to the upper end of the central opening of the matrix holder. Preferably, the well strip comprises cavities, whose number corresponds to the number of matrix holders on a support. Alternatively, the well strip can comprise more cavities than matrix holders contained on a support, and/or at least one of the cavities of the well strip can take up more than one matrix holder. Optionally, the well strip comprises only one cavity, which is large enough to take up all matrix holders of a support or of several supports. If a cavity of the well strip comprises several matrix holders, the matrix holders are in contact with the same medium, which can avoid for example variations in the composition of the medium between different cavities.

According to a particular embodiment, the well strip comprises an element for fixed or reversible attachment to the transport unit. Optionally, the well strip also comprises an abrasive or stripping construction. An abrasive construction is particularly useful when using the cell culture device in an automated environment, for example with a robot, as this way the dripping of cell culture medium during transport of individual units can be prevented.

According to a particular embodiment, at least one of the cavities comprises an insert, thereby reducing the volume of the cavity. For example, this can reduce the volume of cell culture medium or hydrogel required, thereby significantly reducing the costs of the cell culture. By reducing the volume, also the amount of reagents tested or used on the cells in the cell culture system can be reduced.

The components of the present cell culture device can comprise any material that can be used in a cell culture system. In particular, the components of the cell culture device comprise biocompatible and sterilizable material. This includes for example materials such as silicone, polycarbonate, polystyrene, polyethylene, polysulfone (PSU), polyphenylsulfone (PPSU), and other materials commonly used in cell culture systems.

The matrix described herein can be a solid porous support or a hydrogel, or a combination thereof. For example, the matrix can be a solid porous support, to which a hydrogel is applied and/or which is filled with a hydrogel. For example, the porous matrix can also be dehydrated. Specifically, the porous matrix can be a poly(L-lactide) (PLLA), processed into microfibers, which is very consistent in terms of fiber diameter and pore size. Alternatively, it can be polystyrene scaffolds suitable for 3D cell culture. In particular, the matrix consists of natural, semi-synthetic or synthetic material, or a combination thereof. Optionally, the matrix can be modified or coated with proteins or peptides. For example, the matrix can comprise antibodies, antibody fragments, or RGD sequences. RGD sequences are amino acid sequences containing arginine/glycine/asparagine. These sequences occur in proteins of the EC matrix, for example fibronectin/vitronectin. Cells can bind to them via integrins.

The porous matrix can be transferred into the matrix holder, alternatively it can also be integrated into the matrix holder via lyophilization.

In particular, any material which is compatible with the culture of animal cells, can be used as material for the matrix described herein. In particular, the natural material is selected from collagen, fibrin, glycosamine glycans, and hyaluronic acid, and the synthetic or semi-synthetic material is selected from polymeric components such as polymethyl methacrylate, polylactic acid, polyglycol, polystyrene and ceramic components such as hydroxyapatite or tricalcium phosphate.

According to a particular embodiment, the matrix in the matrix holder is prepared with proteins or protein components. Thereby, the hydrogel or the solid porous support is formed in the matrix holder. Subsequently, the matrix can be functionalized with functional proteins such as antibodies, growth factors or extracellular protein as well as with functional peptides by traditional cross-linking techniques such as NHS-EDC or click chemistry or other variants, wherein the different fluids are stored in different well strips and the chemical reactions take place via manual change from one well strip to the next. Afterwards can follow a colonialization with cells or surgical intervention in an animal organism.

In the present cell culture system, adherent cells as well as suspension cells can be cultivated.

The cells to be cultivated are preferably eukaryotic cells, in particular animal cells. Preferably the cells are mammalian cells, specifically preferred human but also non-human, such as rodent cells for example from mouse, hamster, opossum, potorous, or rat, insects, bovine cell lines, dog such as MDCK cell lines, pig, etc.

The cells can be stem cells, e.g. omnipotent, pluripotent, multipotent or unipotent, or differentiated tissue cells or blood cells or lymphocytes. The cells can be from immortalized cell lines, e.g. tumor cell lines. It is possible that the cells are cultivated in such a way that differentiation takes place. Thus, cells can also change the degree of differentiation. This should preferably be determined by the culture conditions (e.g. growth or differentiation factors).

Adherent cells comprise any animal, in particular human, cells, typically used in biomedical or pharmaceutical research. These include for example stem cells from diverse sources such as the mesenchyme, fat, liver, brain or other sources or differentiated cells such as fibroblasts, chondrocytes, osteoblasts, epithelial cells of different origins, endothelial cells, neuronal cells, hepatocytes as well as induced pluripotent stem cells of embryonic origin, and cell lines.

Suspension cells can comprise for example blood precursor cells or differentiated blood cells. Thereby, suspension cells can either be embedded in a hydrogel matrix or cultivated as a suspension in the well strip as part of co-culture studies, wherein in the latter case an adherent cell culture is embedded in a matrix in the matrix holder. For example, interaction studies can be performed between blood cells as suspension and mesenchymal stem cells in a hydrogel in the matrix holder.

A three-dimensional cell culture system refers to the cultivation of cells in a microstructured three-dimensional cell culture under in vitro conditions. The culture or its cells shall adopt a spatial orientation. This happens mainly in the form of hydrogels made of scaffold proteins such as fibrin, collagen, gelatine(poly)methacrylate or matrigel as well as solid scaffolds such as polystyrene polylactate acid or other chemical substances. In a three-dimensional environment, many cell lines form spheroids, whose diameter increases over time after embedment of the cells. Also non-spheroid forming cells often show a morphology, which is very different from 2D cultures.

Preferably, in the cell culture device according to the invention and in the method according to the invention, a cell-compatible liquid is used, in particular cell culture medium. In this liquid the above described steps ii) to v) are preferably performed. This liquid is used as part of the complex cellular environment. It can be a medium suitable for cell growth or cell supply. Defined cell culture media are based on the component groups amino acids, carbohydrates, inorganic salts and vitamins. Often included salts are for example calcium chloride, potassium chloride, magnesium sulfate, sodium chloride and monosodium phosphate. Often included vitamins are for example folic acid, nicotinamide, riboflavin and B12. In addition, the cell culture medium can contain FCS (fetal calf serum) or FBS (fetal bovine serum). Preferred cell culture media include MEM, α-MEM, DMEM, RPMI and variations or modifications thereof.

If the cell culture device according to the invention also comprises a cell-compatible liquid, such as cell culture medium, and living cells, it is also referred to as a cell culture system.

In particular, the cell culture system comprises the container and the culture medium, but not the specific atmospheric composition. Thus, in step ii) of the present method, the cells can be removed from the incubator, in which atmospheric conditions are present, which are intended to promote the best possible growth. The preferred atmospheric conditions for the best possible cell growth for animal cells are 35-38° C., particularly preferred about 37° C., and 0-10% CO₂, particularly preferred 3-6% CO₂.

A transport unit described herein for transport of the cell culture units comprises preferably a support rail (22) for fastening one or more cell culture units, a uptake unit (20a) for a support rail (22), a cover (23), and two spacer elements (24), which are optionally directly connected to each, for uptake in an orderly manner of at least two or more supports (10) of a matrix holder. Optionally, the transport unit can comprise one or more identification elements (26) such as an RFID, barcode or QR code, and/or a centering unit (27), especially for automated processes. The transport unit consists preferably of a solid material, for example a plastic, which is preferably temperature resistant and/or acid resistant. The single elements of the transport unit can be made of the same material or of different materials.

Optionally, the transport unit described herein can further comprise a plate (48) which forms the underside of the transport unit, openings in the plate (48) for centering the transport unit on the plate (49), openings in the plate (48) for screw fittings (50), a further plate (51) which is mounted approximately at the middle height of the transport unit, magnetizable metal bars (52), a centering holder (54), openings in the plate (51) for the centering holder (54), and/or a centering object (55).

The magnetizable metal bars (52) allow a reversible connection to an external construction, such as a robot, which is used for the automated manipulation of the cell cultures. For example, the magnetizable metal bars (52) serve as a docking point for reversible connection to a robotic unit for transport of the transport unit, for example for reversible connection to magnets of the Oli-MAT (available from LifeTaq-Analytics GmbH, Tulln an der Donau). The Z-axis of the Oli-MAT comprises two permanent magnets, with whose help the transfer unit can be carried from position to position. By means of a short impulse, the magnets can be demagnetized for a short time, which leads to the stopping of the transfer unit.

In addition, the system includes further one device each for colonialization in horizontal and in vertical orientation.

In the case of vertical colonialization, the cells are mixed for example with a hydrogel and transferred to the device for vertical colonialization, a well strip, by pipette. Subsequently, the matrix holder with the matrix holder device is immersed in the hydrogel until it polymerizes. Afterwards the matrix holder is carefully transferred into a well strip filled with medium, wherein only a defined amount in the inner opening of the polymerized hydrogel is transferred along with corresponding cells. Depending on the concentration of the hydrogel components, the polymerization can take a few minutes up to hours. Accordingly, the culture has to be transferred to an incubator if necessary. To avoid air bubbles in the opening during polymerization, the matrix holder in this case can have a small opening in the vertical surface, above the opening.

In another variant of the vertical colonialization, the matrix holder is first transferred to the vertical colonization device and subsequently the hydrogel with cells is added. After polymerization, again transfer into a well strip filled with medium takes place.

Alternatively, in the case of hydrogels, the colonialization can take place in a horizontal arrangement with a horizontal colonization device. Thereby, the matrix holder with the device for adhering the matrix holder is attached to the colonization device beforehand. Afterwards, mixing of the cells with the hydrogel and transfer of this mixture into the opening of the matrix holder is performed. After polymerization, the device is manually detached from the matrix holder and transferred into a well strip filled with medium. With this arrangement the volume compared to the vertical arrangement can be reduced by at least a factor of 5.

For horizontal colonialization, the colonization device can be provided with a plateau. After colonialization as described above, the matrix holder is detached from the horizontal colonization device, wherein a cavity on the backside of the hydrogel is formed. In a particular embodiment, colonialization with a further cell type, for example of epithelial origin such as endothelial cells of a blood vessel or epithelial cells of the intestine, is then possible on this cavity and can form a barrier-like structure.

In the case of porous solid supports, the colonialization preferably takes place in a horizontal arrangement. In this case, the central opening comprises a cavity with a wall. On this wall lies the porous support, on which cells can be colonialized directly, whereby in this arrangement the construct can be transferred to an incubator for several hours up to a day. After colonialization is complete, the matrix support can be transferred to a well strip filled with medium. Depending on the support, the support can be dehydrated in several steps prior to colonialization. This can be done in advance by transferring the device with the matrix holder from one liquid-filled well strip to another liquid-filled well strip. After colonialization, a subsequent media change can be performed, by transferring the device with the matrix holder and the three-dimensional cell culture from the old well strip with old medium to a new well strip previously filled with fresh medium. In a particular embodiment, the transfer of the well strips with the matrix holders can also take place automated using the transport unit or a robotic gripper arm.

For long-term cultivation of cells in the matrix as hydrogel or porous degradable support, the culture can be decellularized according to a particular embodiment, to obtain an extracellular matrix free of cells. Hereby for example, mesenchymal stem cells can be differentiated into osteoplasts, chondrocytes or adipocytes over several weeks and finally the obtained matrix can be made cell-free using a standard decellularization protocol.

In another possible variant, a pre-culture in 2D is completely omitted and passaging of the cells in 2D is avoided. In this case, cells are colonialized at low concentration (<5000-10000/well) on the matrix support, either as hydrogel or as solid porous support and cultivated to a high concentration. Subsequently, the matrix is dissolved or the cells are detached from the support by enzymatic reaction and recovered for a next colonialization.

In another embodiment, the matrix holder can be filled with cells and a hydrogel matrix and transferred to a device for perfusion, wherein a continuous or periodic flow can be initiated.

The cell culture device according to the invention can be stored or transported in a box, optionally with a specific insert for uptake of the devices. Specifically, the box can comprise further elements for temperature control or cooling or elements for temperature control and/or temperature recording or sensors for CO₂ or O₂ measurement. Furthermore, the box can be an isothermal box.

The matrix holder, for example made of silicone, can alternatively be transferred to a bioreactor system with perfusion. For example, cells can be grown at low concentration, when the cell number becomes too large for a purely static culture, the matrix holder can be transferred to a larger vessel or bioreactor system with perfusion. This allows initiation of a continuous or periodic flow.

Different methods for use of the cell culture device according to the invention are described herein. For this purpose, the matrix holder can be horizontally or vertically oriented. Specifically, the elements used for cultivation are sterilized, in particular autoclaved. The used cells can be prepared for cultivation, in particular by washing and contact with proteases which degrade those proteins, that maintain the cell association, for example trypsin. This allows the cells to be detached from a support, separated and suspended in the further process. To increase cell density cells can also be centrifuged. The cell number for embedding or colonialization of the matrix can be determined by the person skilled in the art, for example the number of cells can be about 1000-100000 cells per matrix. The matrix holder can be transferred to a well strip (11), in particular using the support of the matrix holder (10). The conditions for the colonialization of the matrix in the opening (13) of the matrix holder (12) with the cell suspension can be adapted individually for the respective cell types, for example the temperature can be about 30° C. to 38° C., for example about 37° C. for mammalian cell cultures.

According to another embodiment, the cell culture device can comprise one or more electrodes. In particular, the electrodes are arranged such that the cells to be cultivated can be electrically stimulated. Preferably, the electrodes are located in the well strip, in particular in one or more of the cavities of the well strip.

The invention further comprises the following embodiments:

1. Cell culture device for a three-dimensional cell culture comprising one or more cell culture units, characterized in that a cell culture unit (21) comprises:

i. at least one matrix holder (12), with a central opening (13) for uptake of a matrix;

ii. a support (10), which is fixedly or reversibly connected to the at least one matrix holder (12); and

iii. a well strip (11), consisting of a vessel with at least one cavity, which can comprise at least one matrix holder (12) up to the upper end of the central opening (13) or more,

wherein the matrix holder (12) is vertically oriented.

2. The cell culture device according to point 1, wherein the central opening (13) of the matrix holder (12) is a continuous opening (13a and 13c) or a cavity with a wall (13b).

3. The cell culture device according to point 1 or 2, wherein the central opening (13) comprises a matrix, which is a solid porous support or a hydrogel, or a combination thereof.

4. The cell culture device according to any one of points 1 to 3, wherein the matrix consists of natural, semi-synthetic or synthetic material, or a combination thereof.

5. The cell culture device according to point 4, wherein the natural material is selected from collagen, fibrin, glycosamine glycans and hyaluronic acid, and the synthetic or semi-synthetic material is selected from polymeric components such as polymethyl methacrylate, polylactic acid, polyglycol, polystyrene and ceramic components such as hydroxyapatite or tricalcium phosphate.

6. The cell culture device according to any one of points 1 to 5, wherein the support (10) comprises an element (15) for uptake of the matrix holder (12) and the matrix holder comprises notches (14a, 14b) at its upper end, via which it can be reversibly connected to the element (15) of the support (10).

7. The cell culture device according to any one of points 1 to 6, wherein the device comprises at least 5, in particular at least 10 matrix holders (12).

8. The cell culture device according to any one of points 1 to 7, wherein the device additionally comprises a transport unit (20), which can transport one or more cell culture units (21).

9. The cell culture device according to point 8, wherein the transport unit (20) comprises

i. a support rail (22) for attachment of one or more cell culture units (21) to the transport unit (20),

ii. an uptake unit (20a) for a support rail (22),

iii. a cover (23), and

iv. two spacer elements (24), which are optionally directly connected to each other, for uptake in an orderly manner of at least two or more supports (10) of a matrix holder,

v. optionally at least one identification element (26) such as for example a RFID chip or a barcode, and

vi. optionally a centering unit (27).

10. The cell culture device according to point 8 or 9, wherein the well strip (11) comprises a position (25) for attachment to the support rail (20).

11. The cell culture device according to point 8 or 9, wherein the cell culture units are arranged in parallel in the spacer (24).

12 Method for cell cultivation using the cell culture device according to any one of points 1 to 11.

13. Method according to point 12, comprising the steps

i. suspending the cells to be cultivated in an appropriate amount of a cell culture medium to form a cell suspension,

ii. colonializing the matrix in the opening (13) of the matrix holder (12) with the cell suspension,

iii. transferring the matrix holder into a well strip (11), in particular using the support of the matrix holder (10), and

iv. incubating the cell culture device, which contains the cells, under conditions that allow cell growth.

14. Method according to point 13, wherein the colonialization of the matrix with the cell suspension takes place in horizontal arrangement of the matrix holder (12).

15. Method according to item 14, wherein the opening (13) of the matrix holder (12) comprises a cavity (13c) for attachment and is reversibly connected to a colonization device (30) via a complementary sealing element (31), and the matrix is a hydrogel, comprising the steps

i. suspending the cells to be cultivated in an appropriate amount of a cell culture medium to form a cell suspension,

ii. mixing the cell suspension with one or more components of a hydrogel, to produce a cell suspension-hydrogel mixture,

iii. colonializing the opening (13) of the matrix holder (12), which is horizontally aligned in the colonization device (30), with the cell suspension-hydrogel mixture,

iv. incubating the cell batch in a suitable cultivation environment, preferably at 37° C. and 5% CO₂, until polymerization of the matrix,

v. separating the matrix holder (12) from the colonization device (30)

vi. transferring the matrix holder (12) into a well strip (11) filled with medium, preferably using the support of the matrix holder (10),

vii. incubating the cell culture device, which contains the cell suspension-matrix mixture, under conditions that allow cell growth.

16. Method according to point 13, wherein the colonialization of the matrix with the cell suspension is performed in a vertical arrangement of the matrix holder (12).

17. Method according to point 16, wherein the opening (13) of the matrix holder (12) comprises an upper opening (13d) and the matrix holder (12) is reversibly connected to a colonization device (33), and the matrix is a hydrogel, comprising the steps

i. suspending the cells to be cultivated in an appropriate amount of a cell culture medium to form a cell suspension,

ii. mixing the cell suspension with a hydrogel, to produce a cell suspension-hydrogel mixture,

iii. filling the wells of the colonization device (33) with the cell suspension-hydrogel mixture,

iv. colonializing the opening (13) of the matrix holder by immersing the matrix holder (12) in the colonization device (33),

v. incubating the cell batch in a suitable cultivation environment, preferably at 37° C. and 5% 5% CO₂, until polymerization of the matrix,

vi. transferring the matrix holder (12) from the colonization device (33) into a well strip (11) filled with culture medium, and

vii. incubating the cell culture device, which contains the cell suspension-matrix mixture, under conditions that allow cell growth.

18. Method according to point 16, wherein the opening (13) of the matrix holder (12) comprises an upper opening (13d) and the matrix holder (12) is reversibly connected to a colonization device (33), which comprises filling elements (40), and the matrix is a hydrogel, comprising the steps

i. suspending the cells to be cultivated in an appropriate amount of a cell culture medium to form a cell suspension,

ii. mixing the cell suspension with a hydrogel, to produce a cell suspension-hydrogel mixture,

iii. inserting the matrix holder into the colonization device (33),

iv. filling the wells of the colonization device (33) with the cell suspension-hydrogel mixture via the filling elements (40), thereby colonization the opening (13) of the matrix holder with the cell suspension-hydrogel mixture,

v. incubating the cell batch in a suitable cultivation environment, preferably at 37° C. and 5% 5% CO₂, until polymerization of the matrix,

vi. transferring the matrix holder (12) from the colonization device (33) into a well strip (11) filled with culture medium, and

vii. incubating the cell culture device, which contains the cell suspension-matrix mixture, under conditions that allow cell growth.

19. Cell culture device according to any one of points 1 to 7, wherein the well strip is a perfusion device (34), and the perfusion device comprises tube connections (41), optionally for connection to a pump.

20. Cell culture device according to any one of points 1 to 7, further comprising a box (44) and an insert (42) for uptake of one or more cell culture units (21) in the box (44).

21. Cell culture device according to any one of points 1 to 7, additionally comprising a box (44) and an insert (43) for uptake of one or more colonization devices (30).

### EXAMPLES

The present invention is further described by the following examples, without necessarily being limited to these specific embodiments of the invention.

### Example 1

Colonializing a Hydrogel Matrix with Cells Using the Horizontal Colonization Device (30) and Subsequent Cultivation of the Cells in a Cell Culture Unit

The support with the matrix holders (13c) was connected to the colonization device (30) and autoclaved together. Subsequently, a 2D culture of mesenchymal stem cells was washed 2× with DPBS without Ca/Mg and trypsinized. The solubilized cells were then taken up in medium with serum and transferred to 50 ml Falcon. Next, these were centrifuged by ultracentrifuge at 0.3 RPM 5 min and the supernatant discarded. In the next step, the addition of new medium without serum took place and furthermore a cell counting was performed. Subsequently, the cells were centrifuged again at 0.3 RPM for 5 min and the supernatant discarded. For 50 matrix holders a 5 ml cell-fibrinogen-thrombin solution was prepared. Depending on the initial concentration of fibrinogen and thrombin different volumes are used. In the present example, the cell pellet was taken up in 1.75 ml medium without serum, so that a cell number between 1000-100000 cells per matrix was incorporated. Afterwards the addition of 750 μl of reconstituted fibrinogen took place, so that a concentration of 5 mg/ml of fibrinogen was achieved. Next, 2 μl of a thrombin stock solution (8000 U/ml) was transferred to 250 μl of 40 mM CaCl₂) (˜64 U/ml). From this solution, 40 μl were transferred into 2.5 ml of 40 mM CaCl₂) (˜1 U/ml). 45 μl of the cell-fibrinogen solution was pipetted into a matrix holder (12) with a pipette. Subsequently, 45 μl of thrombin solution (target concentration fibrinogen ˜2.5 mg/ml thrombin 0.5 U/ml) was carefully added to each cell-fibrinogen droplet without air bubbles and transferred to an incubator at 37° C. with 5% CO₂ for polymerization. After polymerization, the colonization device (30) was manually detached from the matrix holder (12) and the support with the polymerized matrix-cell solution was transferred into a new well strip (11) with each ˜1˜1.5 ml of fresh medium and transferred to an incubator (37° C./5% CO₂).

### Example 2

Colonializing a Hydrogel Matrix with Cells Using the Vertical Colonization Device (33) and Subsequent Cultivation of the Cells in a Cell Culture Unit

The colonization device (33) and a support with the matrix holders (13a) were autoclaved together. A 2D culture of mesenchymal stem cells was washed 2× with DPBS without Ca/Mg and trypsinized. The solubilized cells were then taken up in medium with serum and transferred to 50 ml Falcon. Afterwards followed a centrifugation using ultracentrifuge at 0.3 RPM for 5 min and the supernatant was discarded thereafter. In the next step, the addition of new medium without serum took place and a cell counting was performed. Subsequently, the cells were centrifuged again at 0.3 RPM for 5 min and the supernatant discarded again. For 5 matrices, 5 ml of cell-fibrinogen-thrombin solution was prepared. Depending on the initial concentration of fibrinogen and thrombin different volumes are used. In this case, the cell pellet was taken up in 1.75 ml of medium without serum, so that a cell number between 50000-250000 cells per matrix could be incorporated. Afterwards followed the addition of 750 μl of reconstituted fibrinogen, so that a concentration of 5 mg/ml of fibrinogen was achieved. Subsequently, 2 μl of a thrombin stock solution (8000 U/ml) was transferred to 250 μl of 40 mM CaCl₂ (˜64 U/ml). From this solution, 40 μl were transferred into 2.5 ml of 40 mM CaCl₂ (˜1 U/ml). Next, the fibrinogen-cell solution was mixed with the thrombin solution and 0.5 ml-1 ml was transferred to the colonization device (33). Once the solution was in the colonization device, the support was quickly immersed in the matrix holder and the colonization device was stored in an incubator at 37° C. and 5% CO₂ until polymerization.

After polymerization was complete, the support was carefully pulled up from the colonization device and transferred into a new well strip with fresh medium and transferred to an incubator (37° C./5% CO₂).

### Example 3

Colonializing a Solid Porous Matrix and Subsequent Cultivation of the Cells in a Cell Culture Unit

A support with the matrix holders without an opening (13b) and several well strips (11) were autoclaved together. In this case, a porous dehydrated matrix (for example “Alvetex” Reprocell or “Mimetix” TheElectroSpinningCompany) was transferred into the matrix holder using tweezers. Alternatively, the matrix could already be integrated into the matrix holder using lyophilization steps. In the next step, the matrix was rehydrated and washed using ethanol according to the manufacturer's instructions. For an efficient handling herein, the used liquids were transferred into several parallel well strips, so that the support of a well strip could be quickly immersed in the other.

A 2D culture of mesenchymal stem cells was washed 2× with DPBS without Ca/Mg and trypsinized. The solubilized cells were subsequently taken up in medium with serum and transferred to 50 ml Falcon. Afterwards followed again a centrifugation using Ultrafuge centrifuged at 0.3 RPM 5 min and subsequent removal of supernatant. In the next step, new medium with serum was added and a cell counting was performed. Afterwards the cells were centrifuged again at 0.3 RPM for 5 min and the supernatant discarded. For 50 matrix holders a 5 ml cell solution was prepared, with the number of cells ranging between 1000-20000 cells per matrix. The support with the matrix holders was placed horizontally and 90 μl of cell suspension was transferred into the opening on the matrix. In the following, the construct was cultivated in an incubator for 12-24 h at 37° C. and 5% CO₂ until attachment of the cells to the matrix. After attachment, transfer of the support to a new well strip with fresh medium took place and its subsequent cultivation in an incubator (37° C./5% CO₂).

1. A cell culture device for a three-dimensional cell culture comprising one or more cell culture units, wherein each cell culture unit (21) comprises:
i. at least one matrix holder (12) with a central opening or cavity (13) for uptake of a matrix;
ii. a support (10) which is fixedly or reversibly connected to the at least one matrix holder (12); and
iii. a well strip (11) consisting of a vessel with at least one cavity, wherein the cavity can retain at least one matrix holder (12) up to at least an upper end of the central opening (13),
wherein the matrix holder (12) is vertically oriented.

2. The cell culture device according to claim 1, wherein the central opening (13) of the matrix holder (12) is a continuous opening (13a and 13c) or a cavity with a wall (13b), wherein the central opening (13) comprises a matrix, and wherein the matrix is a solid porous support, a hydrogel, or a combination thereof.

3. The cell culture device according to claim 2, wherein the matrix consists of natural, semi-synthetic or synthetic material, or a combination thereof, wherein the natural material is selected from the group consisting of collagen, fibrin, glycosamine glycans, and hyaluronic acid, and wherein the synthetic or semi-synthetic material is selected from the group consisting of polymethyl methacrylate, polylactic acid, polyglycol, polystyrene, hydroxyapatite and tricalcium phosphate.

4. The cell culture device according to claim 1, wherein the support (10) comprises an element (15) for connection with the matrix holder (12) and the matrix holder comprises notches (14a, 14b) at its upper end, wherein the matrix holder can be reversibly connected to the element (15) of the support (10) by the notches.

5. The cell culture device according to claim 1, wherein the device comprises at least 5, or at least 10 matrix holders (12).

6. The cell culture device according to claim 1, wherein the device additionally comprises a transport unit (20) which can transport one or more cell culture units (21).

7. The cell culture device according to claim 6, wherein the transport unit (20) comprises:
i. a support rail (22) for attachment of one or more cell culture units (21) to the transport unit (20),
ii. an uptake unit (20a) for the support rail (22),
iii. a cover (23), and
iv. two spacer elements (24), wherein the spacer elements are optionally directly connected to each other for uptake in an orderly manner of at least two or more supports (10) of a matrix holder,
v. optionally at least one identification element (26), the identification element being a RFID chip or a barcode, and
vi. optionally a centering unit (27).

8. The cell culture device according to claim 6, wherein the well strip (11) comprises a position (25) for attachment to the support rail (20), or wherein the cell culture units are arranged in parallel in the spacer (24).

9. The cell culture device according to claim 6, wherein the transport unit (20) comprises:
i. a first plate (48) forming the underside of the transport unit,
ii. openings in the first plate (48) for centering the transport unit on the plate (49),
iii. openings in the first plate (48) for screw fittings (50),
iv. a second plate (51) mounted approximately at a middle height of the transport unit,
v. magnetizable metal bars (52),
vi. a centering holder (54),
vii. openings in the second plate (51) for the centering holder (54),
viii. and a centering object (55).

10. A method for cell cultivation, comprising:
i. suspending the cells to be cultivated in an appropriate amount of a cell culture medium to form a cell suspension,
ii. introducing the cell suspension into the cell culture device according to claim 1,
iii. incubating the cell culture device under conditions that allow cell growth, wherein the cell culture device contains the cell suspension.

11. The method according to claim 10, further comprising:
i. colonizing the matrix in the opening (13) of the matrix holder (12) with the cell suspension by introducing the cell suspension into the cell culture device according to, and
ii. transferring the matrix holder into a well strip (11) using the support of the matrix holder (10).

12. The method according to claim 11, wherein the opening (13) of the matrix holder (12) comprises a cavity (13c) for attachment and is reversibly connected via a complementary sealing element (31) with a colonization device (30), and the matrix is a hydrogel, further comprising:
i. mixing the cell suspension with one or more components of a hydrogel, to produce a cell suspension-hydrogel mixture,
ii. colonizing the opening (13) of the matrix holder (12), which is horizontally aligned in the colonization device (30), with the cell suspension-hydrogel mixture,
iii. incubating the cell batch in a suitable cultivation environment at 37° C. and 5% CO2, until polymerization of the matrix,
iv. separating the matrix holder (12) from the colonization device (30), and
v. transferring the matrix holder (12) into a well strip (11) filled with medium, preferably using the support of the matrix holder (10).

13. The method according to claim 12, wherein the opening (13) of the matrix holder (12) comprises an upper opening (13d) and the matrix holder (12) is reversibly connected to a colonization device (33), and wherein the matrix is a hydrogel, further comprising:
ii. filling the wells of the colonization device (33) with the cell suspension-hydrogel mixture,
iii. colonizing the opening (13) of the matrix holder by immersing the matrix holder (12) in the colonization device (33), and
iv. incubating the cell batch in a suitable cultivation environment, preferably at 37° C. and 5% CO2, until polymerization of the matrix.

14. The method according to claim 12, wherein the opening (13) of the matrix holder (12) comprises an upper opening (13d) and the matrix holder (12) is reversibly connected to a colonization device (33), wherein the colonization device comprises filling elements (40), and the matrix is a hydrogel, further comprising:
i. inserting the matrix holder into the colonization device (33), and
ii. filling the wells of the colonization device (33) with the cell suspension-hydrogel mixture via the filling elements (40), thereby colonizing the opening (13) of the matrix holder with the cell suspension-hydrogel mixture.

15. The cell culture device according to claim 1, wherein the well strip is a perfusion device (34), and the perfusion device comprises tube connections (41), optionally for connection to a pump.

16. The cell culture device according to claim 1, further comprising a box (44) and an insert (42) for uptake of one or more cell culture units (21) or colonization devices (30).
